(19) United States
(12) Patent Application Publication
(10) Pub. No.: US 2005/0288793 A1
(43) Pub. Date: Dec. 29, 2005

(54) Internal fixation element for hip acetabular shell

(75) Inventors: Nicholas Nai Guang Dong, Little Falls, NJ (US); Russell Don Meldrum, Zionsville, IN (US); Viktor Erik Krebs, Rocky River, OH (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 10/878,787

(22) Filed: Jun. 28, 2004

(51) Int. Cl. A61F 2/32

(52) U.S. Cl. 623/22.28; 623/22.32

(57) ABSTRACT

An acetabular cup assembly for a prosthetic hip joint includes an outer shell for attachment to the acetabulum. The outer shell has an inner surface receiving a bearing insert portion. Complimentary coupling features are provided on the inner surface of the outer shell and the outer surface of the bearing insert for coupling the bearing insert to the outer shell. A plurality of adaptors are provided having an outer surface for engaging an inner surface of the outer shell and having an inner surface for receiving the bearing insert. Each adaptor has at least one radial extending strap or flange for engaging the bone surrounding the acetabulum. The straps or flanges may be of different sizes and shapes and may be at different locations on the adapter. The inner surface of the adaptor includes the same coupling features as the shell so as to be able to receive a bearing insert.

### BACKGROUND OF THE INVENTION

This invention relates generally to orthopedics and, in particular, to a versatile reconstruction system associated with an acetabular prosthetic implant.

The replacement of members of a natural hip joint with prosthetic implants has become widespread and is being accomplished with ever-increasing frequency. The variety of conditions encountered when effecting such implants has led to the use of various bearing materials and attachment methods placed at an optimum position and orientation, as determined by conditions encountered at the site of the implant. The choice of a particular material for the bearing, as well as the size, positioning and orientation of the bearing member, is determined by the surgeon performing the procedure. Usually such choices are made on the basis of a pre-operative assessment of the needs of a particular patient; however, at times the choices are not completed until the implant site actually is being prepared and conditions encountered at the site can be evaluated during the implant procedure itself. Accordingly, it would be advantageous to have available a greater range of interoperative choices, as well as pre-operative choices, so as to enable a surgeon to accommodate the needs of a particular patient as determined by either or both a pre-operative assessment and an evaluation of conditions encountered at a particular implant site, and to do so in a practical manner.

Especially in the case of revision surgical procedures wherein the pelvis has been severely compromised or deteriorated, it is known to use support structures to receive an acetabular prosthetic device. More particularly, this invention relates to optional modular adaptors which include wings, straps or flanges to enhance the support of the acetabular prosthesis on the pelvis.

One such structure is disclosed in U.S. Pat. No. 5,314,490 to Wagner et al., entitled OUTER CUP FOR AN ARTIFICIAL HIP JOINT SOCKET. According to this patent, an artificial hip joint socket for fastening to a pelvic bone includes a metallic outer cup forming a concavity for receiving a hip, which terminates in an equatorial edge to which supporting flaps are fastened. The flaps include holes to receive bone screws and have preset lines or grooves to enabling preferential bending to provide conformance with the pelvic region surrounding the procedure.

U.S. Pat. No. 5,425,778 relates to an acetabular socket supporting ring having at least one attaching strap for attachment to a corresponding bone portion. This construction has a disadvantage that the support ring must be used in all applications since it receives the outer shell of the acetabular prosthesis.

U.S. Pat. Nos. 5,871,548 and 6,340,370 disclose modular outer shells having wings, straps or flanges which selectively attach to the shell if required.

The present invention provides the surgeon with the ability to choose, either pre-operatively or interoperatively, an optimum, position and orientation for an acetabular cup assembly to be implanted at a particular implant site, with increased ease and at lowered expense. As such, the present invention attains several objects and advantages, some of which are that it allows the choice of the size, position and orientation of the bearing surface of a bearing member selected for assembly with a particular acetabular shell; increases the range of the bearing size, positioning and orientation, and renders the choices available in a practical manner for either pre-operative or interoperative selection; allows a surgeon greater latitude in accommodating the needs of different patients while meeting the requirements imposed by various conditions encountered at a particular implant site, and enables appropriate choices to be made interoperatively, as well as pre-operatively; promotes greater accuracy in the replacement of a natural hip joint, with increased economy; provides a surgeon with the ability to make both pre-operative choices and interoperative choices from a wider range of options; facilitates the insertion and securement of a selected bearing member within an acetabular shell in appropriate alignment and orientation of the bearing member within the acetabular shell; provides an acetabular cup assembly having accurate sizing, positioning and orientation, with economy of manufacture and use, and long-term reliability.

### SUMMARY OF THE INVENTION

The above objects and advantages, as well as further objects and advantages, are attained by the present invention which may be described briefly as an acetabular cup assembly for receiving a proximal end of a femoral component of a prosthetic hip implant, the femoral component including a head member and a neck member depending from the head member, the acetabular cup assembly having an external shell member with an internal cavity, including a coupling element and an internal bearing member for securement within the cavity to receive the head member of the femoral component for rotational movement within the bearing member, the internal bearing member has an external coupling element and may be selected from a plurality of bearing members which may have different characteristics. The cup assembly optionally includes a metallic securing adaptor having wings, flanges or straps extending radially outwardly therefrom which adaptor is provided for reception within the cavity of the acetabular shell. The securing adaptor extends between an upper end and a lower end of the shell and includes an external securing element and an internal coupling element for engaging the external coupling element on the bearing member. The wings, flanges or straps may be modular or integral with the adaptor. Preferably, the wings, flanges or straps are ductile enough to be bent or deformed by the surgeon to closely conform to adjacent bones. Alternatively, the wings, flanges or straps may have grooves or thinner areas to aid in bending. The outer shell preferably has an internal locking element for receiving the adaptor and also an internal coupling for the bearing component. The same locking element may be used for both the bearing and the adapter. The preferred internal locking surface on the outer shell and the corresponding outer locking surface on the adaptor are preferably mating locking tapered surfaces. In the preferred embodiment, this tapered surface on the inner surface of the outer shell is in addition to the coupling elements typically provided for locking a polyethylene bearing insert within the shell.

Further, the present invention provides a shell member for use in an acetabular cup assembly having an internal polymeric bearing member for securement within the shell member. The internal polymeric bearing member being selected from a plurality of bearing members having different characteristics such as bearing surface size and inclination. The shell member includes an internal wall of the cavity with a coupling element on the internal wall cavity of the shell member. The coupling element is compatible with the external securing characteristics of at least one of the plurality of internal bearing members. Optionally, a second additional bearing coupling element may be provided within the cavity of the shell member, the second securing element being compatible with the securing characteristics of at least another of the plurality of internal bearing members. The first coupling element and the second optional coupling element being juxtaposed with one another and placed at relative locations such that the effectiveness of each of the first and second securing elements is maintained in the presence of the other of the first and second securing elements, whereby the one and the another of the internal bearing members each is selectable for effective securement within the shell member to complete the acetabular cup assembly.

The coupling element or elements formed on the inner surface of the shell are also present on the inner surface of the adaptor. This allows the adaptor to be mounted on the tapered conical inner surface of the outer shell and in turn lockingly receive the bearing element. The adaptor elements include outwardly extending wings, flanges or straps for engaging the bone adjacent the acetabulum. The wings or flanges include holes to accommodate bone screws which engage the pelvic bone. The straps or wings may be modular and have mounting features, such as screws which engage corresponding mounting features on the securing elements for their assembly to the hemispherical adaptor body. Such features are well known in the prior art. As discussed above, in addition, the flanges or wings may be bendable to allow the surgeon to conform the shape of the wings or straps to the pelvic bone. This may be done, for example, by making the straps or wings of a deformable material such as a ductile metal or making the material cross-section relatively thin throughout its length or at least at selected areas along the length of the wing or strap.

In addition, the present invention includes a kit of component parts for assembling an acetabular cup assembly having an internal bearing member secured within a shell member, the kit comprising: a plurality of bearing members having different characteristics such that the acetabular cup assembly selectively is provided with characteristics corresponding to the characteristics of a selected one of the internal bearing members (preferably made of ceramic or polyethylene); a plurality of shell members comprising: an internal cavity having a tapered inner surface including at least one coupling element with a first coupling element within the cavity of the shell member being compatible with the securing characteristics of at least one of the plurality of internal bearing members; and optionally, a second coupling element within the cavity of the shell member, the second coupling element being compatible with the securing characteristics of at least another of the plurality of internal bearing members; the first and second coupling elements may be juxtaposed with one another and placed at relative locations such that the effectiveness of each of the first and second coupling elements is maintained in the presence of the other of the first and second coupling elements, whereby the one and the another of the internal bearing members each is selectable for effective securement within the shell member as the selected one bearing member to complete the acetabular cup assembly. The tapered inner surface is also for lockingly engaging the adaptor and is in addition to the first and the optional second coupling elements of the outer shell.

The kit also contains a plurality of adaptor elements all having integral or modular wings, flanges or straps around a part spherical dome which may be inserted within the cup outer shell. These adaptors are to be used intra-operatively to allow the surgeon to affix the cup to an acetabulum where the bone loss has occurred. The wings or straps may be longer or shorter and/or located at different circumferential positions on different adaptor elements.

These and other objects of the invention are set forth in an acetabular cup assembly used in a prosthetic hip joint replacement which has an outer shell for attachment to the acetabulum, a bearing insert portion and an adaptor. The outer shell has a part spherical inner surface and coupling mechanisms adapted to receive either a bearing insert portion or the adaptor. The bearing has an external coupling element for engagement with a complementary coupling element on either the internal surface of the shell or the internal surface of the adaptor. The adaptor has an outer surface for engaging the inner surface of the outer shell with its inner surface adapted for receiving the bearing portion. Each adaptor has at least one radially extending strap, flange or wing which either conforms with or can be deformed in a manner to conform with the bone surrounding the acetabulum.

The wing, flange or strap is deformable at least in a direction perpendicular to the radial direction and may be oriented at various angles and extended various lengths with respect to the body of the adaptor. Obviously, the longer the length, the greater engagement with various areas of the pelvis are possible. The wing, strap or flange may include a reduced cross-section to facilitate deformation or may be made of a ductile material. Alternately, the flange can have a groove or series of grooves to facilitate bending.

The engagement or coupling mechanism between the shell and the outer surface of the adaptor is preferably a complementary conically tapered surface. The surface has a tapered angle so that a locking coupling can be formed between the shell and the insert. Optionally, additionally the shell and adaptor can include a threaded coupling which extends in the polar region of either the cup or shell and can threadably couple the shell and adaptor together. Preferably, tightening of the threaded coupling forces locking engagement between the complementary preferably conically tapered surfaces.

The above structure can be supplied in kit form with a variety of shells, bearing inserts, which are preferably made of polyethylene and/or ceramic and adaptors. The adaptors may have one or more wings located in fixed positions around the circumference of the adaptor or, in fact, may be modular and therefore attachable such as by a threaded connector at any desired angular orientation around the circumference of the adaptor. Thus, the parts may be selected intraoperatively. This is especially helpful when the pelvis is deformed or otherwise missing natural bone so that adaptors having straps oriented in various angular locations and of various lengths can be selected from the kit and placed in position usually utilizing screws. The polymeric or ceramic bearing insert can then be coupled to the insert which is, in turn, coupled to the shell in the same manner as would a similar (somewhat large outer dimension) bearing insert be coupled to a shell without an adaptor.

U.S. Pat. No. 6,475,243 B1, the teachings of which are incorporated herein by reference, teaches the use of a metal coupling element to allow the use of a ceramic bearing insert in an outer shell which can also accept a polyethylene bearing insert. As is typical, the polyethylene bearing insert includes a deformable radial protrusion which extends circumferentially around the outer surface of the bearing insert and which engages a locking feature on the inner surface on the outer acetabular shell. The use of a metal coupling element which engages a tapered surfaces on the outer shell and includes a tapered surface on its inner diameter allows a ceramic bearing having a tapered outer surface to be easily placed in an already implanted outer shell. Thus, if the surgeon decides not to utilize a polyethylene bearing insert, he merely needs to engage the metal coupling element within the outer shell and place a ceramic bearing within the coupling element. Complimentary tapered surfaces are used to lock the ceramic bearing within the coupling element. The coupling element and ceramic bearing may be pre-assembled at the factory. Thus, when a flanged adaptor is used, the tapered outer surface of the coupling element engages the tapered inner surface of the flanged adaptor to lock the bearing element within the adaptor which is in turn coupled to the implanted shell. A one size smaller bearing will have to be used when an adaptor is placed between the bearing and the shell.

### BRIEF DESCRIPTION OF THE DRAWINGS

The invention will be understood more fully, while still further objects and advantages will become apparent, in the following detailed description of the preferred embodiments of the invention illustrated in the accompanying drawing, in which:

FIG. 1 is an exploded view of a typical outer acetabular cup shell with a polymeric bearing insert;

FIG. 1A is an exploded view of an outer acetabular cup shell with a ceramic bearing insert with a coupling element between the ceramic bearing and the shell;

FIG. 1B is an exploded isometric view of the acetabular cup assembly of FIG. 1 including a flanged adaptor and shows a polyethylene bearing insert, a flanged adaptor part and an outer shell;

FIG. 1C is an exploded isometric view of the acetabular cup assembly of FIG. 1A including a flanged adaptor and a ceramic bearing insert, a coupling element, a flanged adaptor part and an outer shell;

FIG. 2 is a bottom view of the acetabular cup shown in FIG. 1b in the assembled condition;

FIG. 3 is a cross-sectional view of the flanged adaptor part shown in FIGS. 1B and 1C;

FIG. 4 is enlarged fragmentary view of a portion of the locking portion of the outer shell component as illustrated in FIGS. 1, 1A, 1B, 1C and 2;

FIG. 5 is an enlarged fragmentary view of a portion of the coupling element for a ceramic bearing shown in FIGS. 1A and 1C;

FIG. 6 is an enlarged fragmentary view of the outer shell and polymeric bearing insert shown in FIG. 1 in the assembled condition;

FIG. 7 is bottom plan view of the flanged adaptor shown in FIG. 3;

FIG. 8 is a cross-sectional view of a ceramic bearing, shell and coupling element illustrated in FIGS. 1A and 5 with a ceramic bearing inserted in an outer shell with a coupling element but without a flanged adaptor;

FIG. 9 is an enlarged fragmentary cross-sectional view of a portion of the assembly of FIG. 1B in the assembled condition;

FIG. 10 is a cross-sectional view of an assembled acetabular implant of FIG. 8 including a ceramic cup and coupling element and further including a metal adaptor within an outer metal shell;

FIG. 11 is an isometric view of an adapter having a short radially extending circumferential flange; and

FIG. 12 is an isometric view of the adaptor part including three wings or straps.

### DETAILED DESCRIPTION

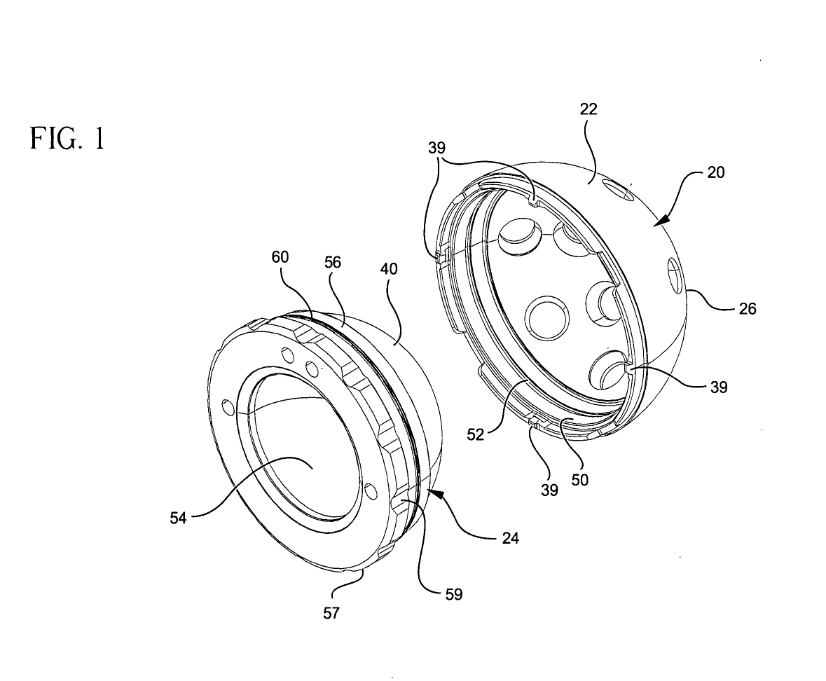

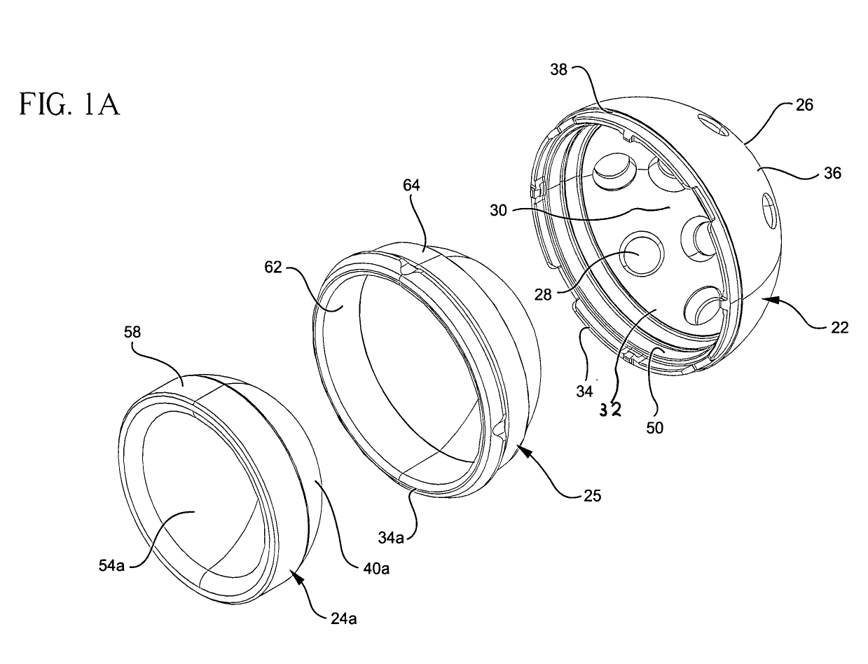

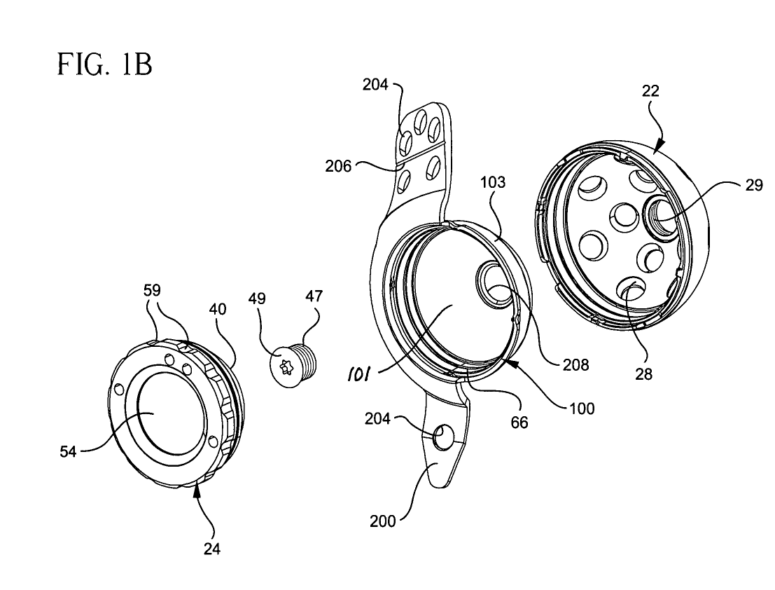

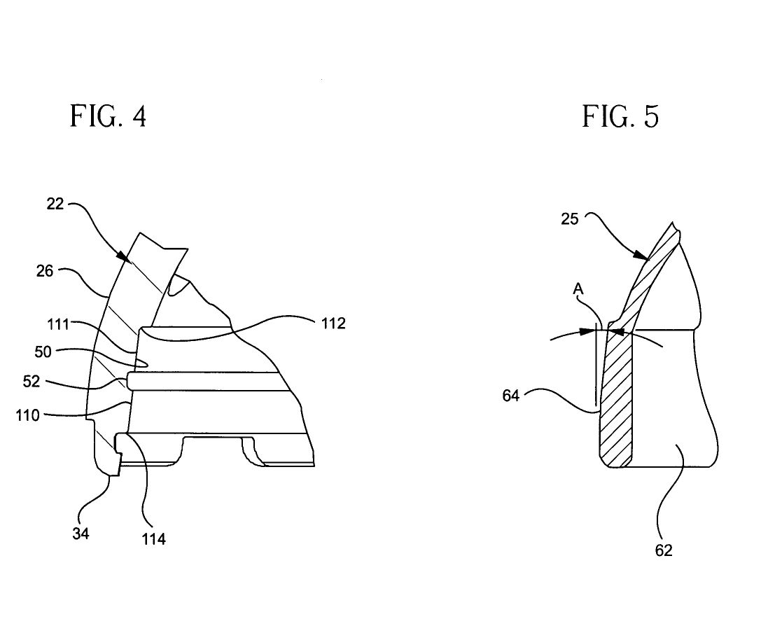

Referring now to the figures, and especially to FIG. 1 there is shown an acetabular cup assembly constructed in accordance with the present invention is illustrated generally as 20. Acetabular cup assembly 20 includes a shell component in the form of metallic shell member 22 and a bearing insert which may be in the form of a plastic (ultra-high molecular weight polyethylene) bearing member 24 as shown in FIG. 1. Alternately, the bearing, as shown in FIG. 1A, may be a ceramic bearing 24a. If a ceramic bearing is used, a metal adaptor 25 may be used (see FIG. 1A). Shell member 22 includes an outer surface 26 having a hemispherical profile configuration which enables shell member 22 to be seated and fixed in place within an appropriately prepared acetabulum in a well-known manner such as by use of bone cement or bone screws. A plurality of screw holes 28 are provided in shell member 22 for receiving anchoring screws (not shown) when such supplemental securing means are desired. As shown in FIG. 1B, a hole 29 is located at the pole of the hemisphere. In the preferred embodiment, hole 29 is threaded. An inner cavity 30 extends upwardly into shell member 22, from a planar lower opening 32 at a lower shell end 34 toward an upper shell end 36. A rim 38 is located adjacent the lower end 34. Rim 38 includes projections 39 extending radially inwardly at one or more locations around the rim surface. As seen in FIG. 4, inner cavity 30 includes a tapered surface portion 50 including a groove or recess 52 located intermediate the ends of the tapered surface portion.

Bearing members 24, 24a have a generally part-spherical domed exterior 40, 40a and a hemi-spherical surface 54, 54a respectively for receiving a spherical femoral head (not shown). The outer surface of the bearing inserts 24, 24a include tapered locking surface 56, 58, respectively, which can engage the tapered inner surface 50 of the shell. The outer surface of polyethylene bearing 24 also includes a deformable ring-like protrusion 60 centered on tapered surface 56. Protrusion 60 lockingly mates with groove 52 of outer shell 22. In addition bearing 24 includes a rim portion 57 with indentations 59 which engage projections 39 to orient a non-symmetrical insert and prevent relative rotation of the bearing within the shell after assembly. Ceramic bearing 24a includes conically tapered circumferential surface 58 but does not have a protrusion similar to 60. In order to use a ceramic bearing with a metal outer shell having a groove coupling system for use with a polymeric bearing insert, it is preferable to use metal coupling element 25 as shown in FIGS. 1A, 1C and 5 to help protect the ceramic bearing member 24a. The ceramic cup 24a has tapered outer circumferential surface 58 adjacent its open end to couple with a mating tapered surface 62 on the inner surface of coupling element 25. These tapered surfaces may be engaged and locked at the factory to protect the ceramic bearing during shipment. Coupling element 25 includes a tapered outer surface 64 to engage the tapered surface on either shell 22, (surface 50) or, as will be described below, on flanged adaptor 100.

Referring to FIG. 1C, there is shown an exploded view of an assembly of an outer shell 22, a flanged insert 100, metal coupling element 25 and ceramic insert 24a. FIG. 1B is similar to FIG. 1C except that a polymeric bearing 24 is used and no adaptor 25 is required. As described in U.S. Pat. No. 6,475,243, a kit can be provided in which various size coupling elements 25 and ceramic inserts 24a can be utilized in connection with outer shell 22. The kit may also include various size polyethylene bearing inserts 24, a wide variety of different size metal adaptor 25 and different size ceramic inserts 24a. A plurality of flanged adaptors 100 of varying sizes can also be included in the kit and thus can be inserted into shell 22 using the tapered interconnections and, if desired, utilizing a threaded screw 47 engaging threaded bore 29 of shell 22. When a ceramic bearing is used with a flanged adapter 100, a coupling element 25 sized to fit within adaptor 100 is used with the conically tapered outer surface 64 of coupling element 25 engaging the inner tapered surface 66 of flanged adaptor 100.

Acetabular cup assembly 20 is to be implanted in stages; that is, the shell member 22 and the bearing member 24, 24a are to be assembled intraoperatively, so as to enable appropriate sizing, placement and orientation of the bearing surfaces 54, 54a, based upon a pre-operative assessment or upon an evaluation of conditions encountered at the site of the implant. To that end, alternate bearing members 24, 24a are made available, in a kit of component parts, which kit provides a plurality of bearing members, the alternate bearing members 24, 24a providing corresponding bearing surfaces 54, 54a which may be placed at different locations and orientations, relative to the already seated and secured shell member 22, any one of which bearing surfaces 54, 54a then being capable of securement in place in the shell member 22, intraoperatively, with the bearing surface 54, 54a appropriately located and oriented for accommodating, in the preferred embodiment, the needs of the patient. If a ceramic bearing is used then coupling element 25 is used.

A selected polymeric bearing member 24 is secured in place appropriately within the shell member 22 via tapered surface elements 56 and protrusion 60 and mating tapered surfaces 50 and groove 62. In addition, in the preferred embodiment, a tapered surface 66 similar to 50 used on shell 22 and also may be located on the inner surface of optional flange adaptor 100 as will be discussed below. If ceramic bearing insert 24a is used then the tapered locking surfaces 62 on the inside of coupling element 25 and outer surface 58 of the bearing 24a hold the bearing 24a in place.

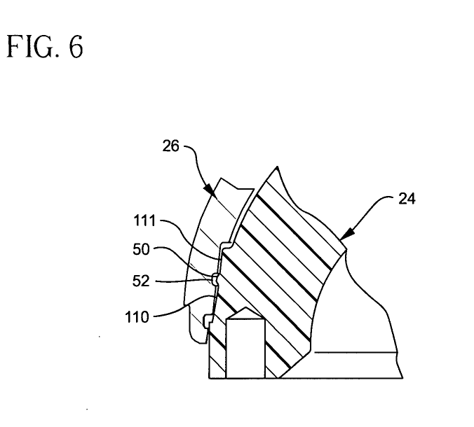

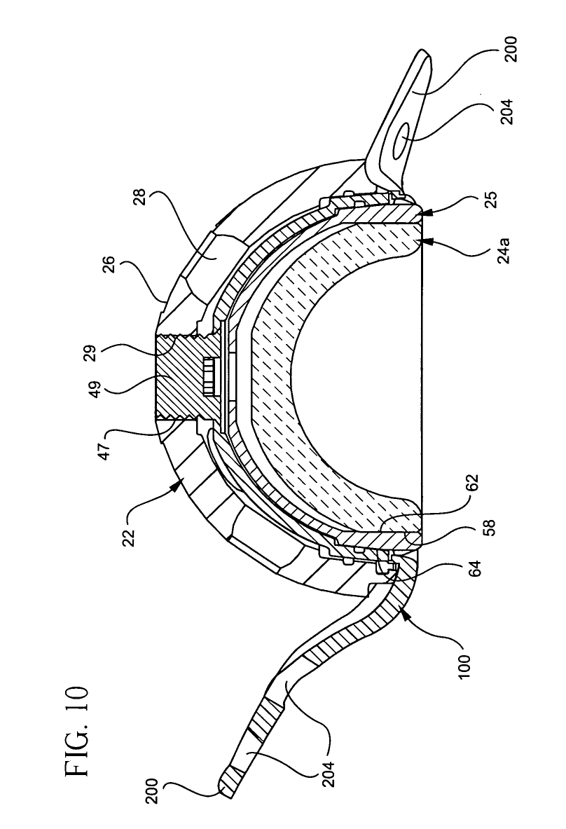

Turning now to FIGS. 1a, 4 and 6, as well as FIG. 10, the preferred coupling mechanism on cup 22 is seen to include tapered surface 50 and an insert securing or locking element in the form of an annular recess or groove 52 extending radially outwardly into the shell member 22 adjacent the lower end 34. A complementary securing element in the form of an annular protrusion or rib 60 extends laterally outwardly from tapered surface 56 of bearing member 24, adjacent the rim 57 of the plastic bearing member 24. A preferred material for the plastic bearing member 24 is an ultra-high molecular weight polyethylene commonly used in connection with such bearing members. The securing characteristics of polyethylene include a resiliency sufficient to assure that upon inserting the bearing member 24 into the shell member 22, as seen in FIGS. 6 and 10, the annular rib 60 is seated within the annular recess 52 of surface 50 to secure the plastic bearing member 24 within the shell member 22.

The position and orientation of the bearing socket 54 relative to the fixed shell member 22 may be selected by providing the different locations and orientations of the bearing socket 54 within the bearing member 24. For example, the orientation of the pole of surface 54 may be angled at various angles with respect to the plane of rim 57, or the depth of bearing surface 54 may vary. Once seated in place, the selected polymeric bearing member 24 is rotationally secured within the shell member 22 by engagement of the complementary securing elements in the form of recess 59 and protrusion 39 and the engagement of tapered surfaces 50 and 56. Protrusion 60 and groove 52 lock the shell and bearing axially. These locking features are provided on all the alternate bearing members 24 (different sizes and bearing socket orientations) in the kit. In addition, once the selected bearing member 24 is seated appropriately within the shell member 22, rotation of the bearing member 24 about the polar axis relative to the shell member 22 is precluded by engagement of protrusions 39 extending radially inwardly from the bottom 34 of the shell member 22 with the counterpart indented portions 59 of the bearing member 24, adjacent the rim or flange 57 of the bearing member 24.

Should the surgeon determine, either on the basis of a pre-operative assessment of a patient or during the course of the implant procedure, that based upon the needs of a particular patient, as determined by the pre-operative assessment or by an evaluation of conditions encountered at the particular implant site, a different bearing material of bearing member 24 would be more appropriate, acetabular cup assembly 20 provides the surgeon with the ability to choose a bearing member having a bearing material which exhibits characteristics more appropriate to the needs of that particular patient such as ceramic bearing 24a. Note bearing 24a, preferably mounted on coupling element 25, can be oriented within the shell 22 prior to being locked by the engagement of tapered surfaces 50 and 64.

Mating tapered surfaces are one of the most effective, convenient, mechanically simple and easily used securement mechanisms available for securing together mechanical components, where the component is constructed of a non-resilient material such as ceramic bearing member 24a. The degree to which the tapered surfaces are tapered depends upon securing characteristics of the particular materials being secured together. Thus, shell 22 has an inner tapered surface 50 to allow for insertion of coupling element 25 which in turn has inner tapered surface 62 for receiving an outer tapered surface 58 on ceramic bearing 24a. In order to enable simplified interoperative securement of a metal flange or strap for engaging the pelvis an adaptor 100 may be placed within shell member 22, subsequent to locating and seating shell member 22 within the acetabulum but prior to insertion of bearing 24 or 24a. The tapered inner surface 50 of shell 22 provides a sample and appropriate way of mating with outer tapered surface 64 of adaptor 100 for mounting the adaptor.

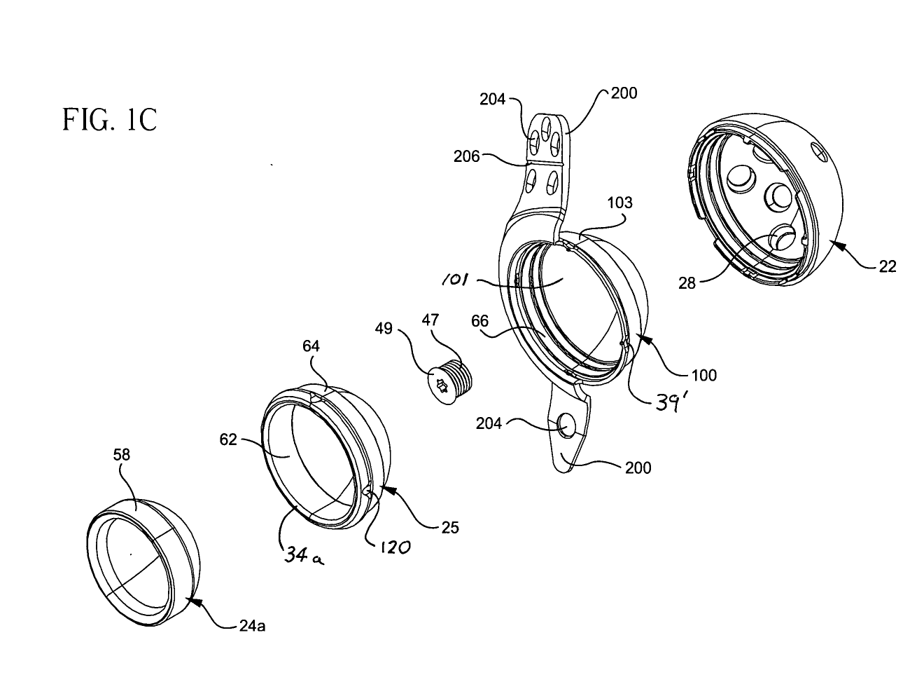

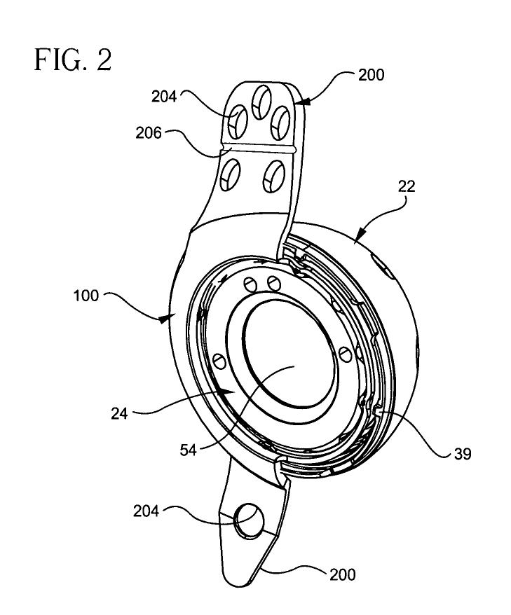

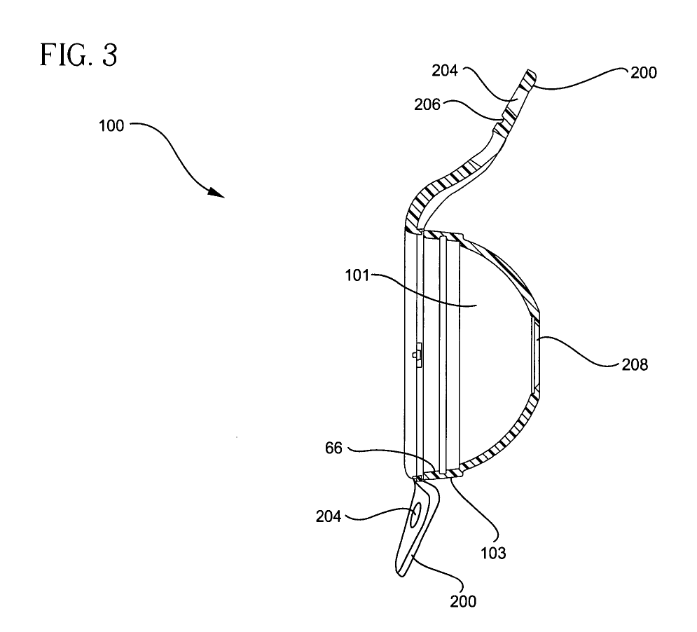

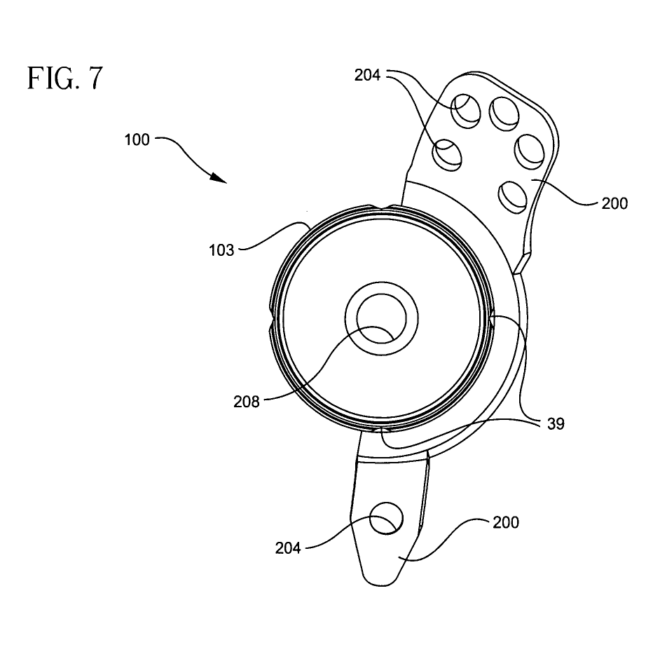

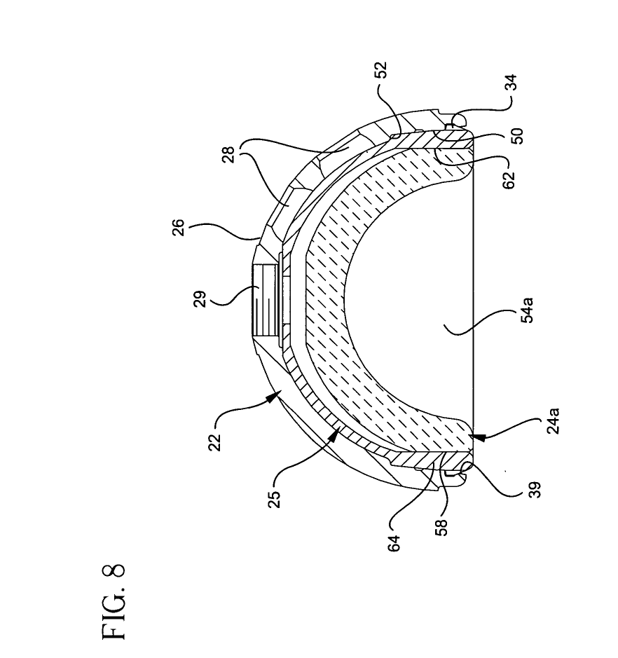

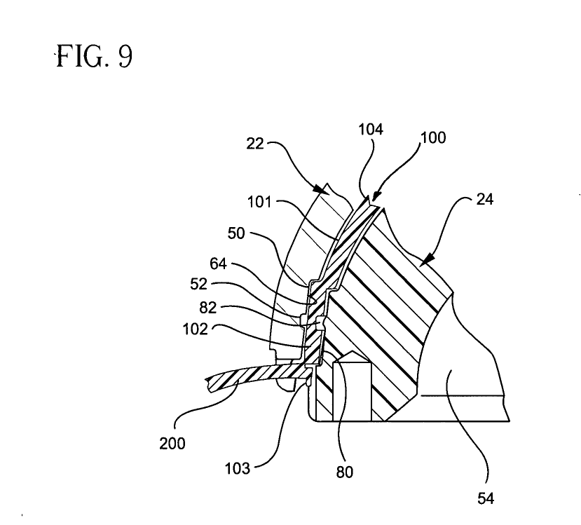

Adaptor 100, shown in FIGS. 1B, 1C, 2, 3, 7 and 9-12 is preferably in the form of a body 101 having an annular ring section 102 adjacent a lower end 103 and a partial domed portion 104 extending between the ring section 102 and an upper end 105. The domed portion 104 is essentially complementary to the counterpart portion of inner cavity 30 of shell 22. The ring portion 102 is provided with respective tapered external and internal securing surfaces in the form of an external tapered seating surfaces 103 and a tapered internal coupling surface 66 having a groove 82 in the same form as the tapered coupling surface 50 and groove 64 of shell 22. The configuration of the internal coupling surface 80 and the configuration of the internal coupling surfaces previously described for shell 22 are compatible with the particular characteristics of the polymeric material of the bearing element 24 so that upon engagement of the external tapered coupling surface 56 of a properly sized bearing 24 with the tapered internal surface of both shell 22 and adaptor cage 100, the bearing member 24 as secured to adaptor 100 or shell 22. Note, however that a one size smaller bearing in the kit is required to mate with adaptor 100 than to mate with shell 22.

As shown in FIGS. 1 and 4, tapered surface 50 of shell 22 further preferably is in the form of two co-linear upper and lower conically tapered seating surfaces 110 and 111. Tapered surfaces 110 and 111 are located in cavity 30 of shell member 22, preferably within the cavity 30 adjacent the lower end 34 of the shell member 22 and separated by groove 52. Conically tapered internal seating surfaces 110 and 111 of tapered surface 50 are complementary to external seating surface of flanged adaptor cage 100 and surface of coupling element 25 for mating engagement of the seating of shell 62. The preferred external seating surfaces 56, 58, 64 and 103 and internal surfaces 50, 62 and 66 are provided with a conically tapered configuration, as illustrated by angle A in FIG. 5, the taper of the configuration being compatible with the securing characteristics of the material of coupling element 25 and flanged adaptor 100 and the shell member 22, which are preferably made of a metal such as titanium. Thus that coupling element 25 and adaptor 100 are secured within the shell member 22 by virtue of the locking of the tapered seating surfaces. In the preferred embodiment, the shell member 22 and coupling element 25 and flanged adaptor 100 are constructed of commercially pure titanium and the complementary angle A is about 12°.

In the preferred embodiment as shown in FIG. 4, conically tapered seating surfaces 110, 111 of shell 22 include an upper end 112 and a lower end 114 and is divided by the recess 52 into upper surface 111 and lower surface 110. By placing the recess 52 essentially midway between the upper edge 112 and lower edges 114, the locking of the seating surfaces 56, 64 and 103 in response to such engagement, is facilitated by virtue of the locking being accomplished along segments 110 and 111 and having generally the same, and therefore maximized, axial length. In this manner, the effectiveness of the seating surfaces 110, 111 in assuring appropriate alignment between the flanged adaptor 100 or coupling element 25 and the shell member 22 as the adaptor or coupling element are inserted into the shell member 22 and in subsequently attaining the desired locking engagement with seating surface 110, 111 is not compromised by the presence of the recess 52.

Should the surgeon determine, either on the basis of a pre-operative assessment of a patient or during the course of the implant procedure, that based upon the needs of a particular patient, as determined by the pre-operative assessment or by an evaluation of conditions encountered at the particular implant site, a winged or flanged adaptor is appropriate because the pelvis has been so severely compromised that additional radial support straps, flanges or wings are required, acetabular cup assembly 20 provides the surgeon with the ability to choose, from a kit of component parts providing a plurality of bearing members 24, 24a and flanged adaptors 100 better meeting the needs of the patient. The surgeon may also choose from a plurality of different polyethylene or ceramic bearing sizes and offset designs which can engage either shell 22 or adaptor cage 100.

If a ceramic bearing 24a is used, it normally comes pre-assembled with coupling element 25 so it that either may be inserted directly into outer shell 22 or into adaptor cage 100. Normally, ceramic bearing 24a ends axially within coupling element 25 above a lower open end surface 34a to protect the ceramic should the neck of the femoral implant, whose head is received within the ceramic bearing, come into contact with metal of the coupling element 25. In addition, the use of coupling element 25 allows, during revision surgery, the easy removal of the ceramic bearing and replacement thereof. Instruments can be provided to grip the metal and break the taper lock between surfaces 64 of coupling element 25 and either tapered surface 52 of shell 22 or surface 103 of adaptor cage 100. It should be noted that the axial locking of the polymeric bearing insert 24 does not depend on the engagement of the tapers but rather the engagement of protrusion 60 and groove 52 of shell 22 or groove 82 of adaptor 100.

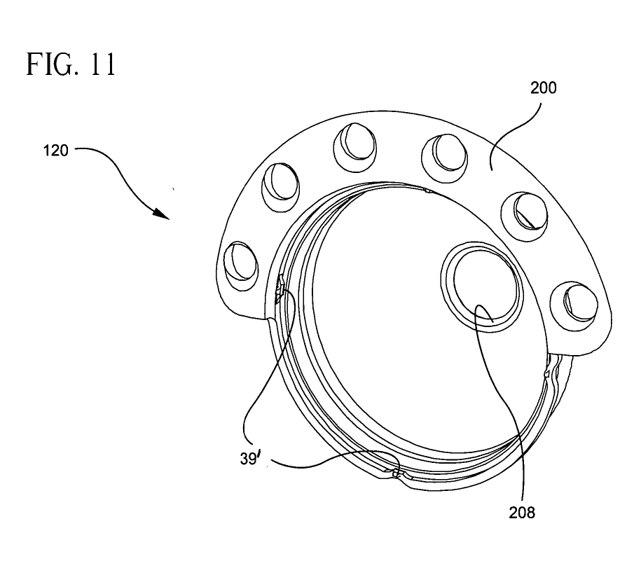

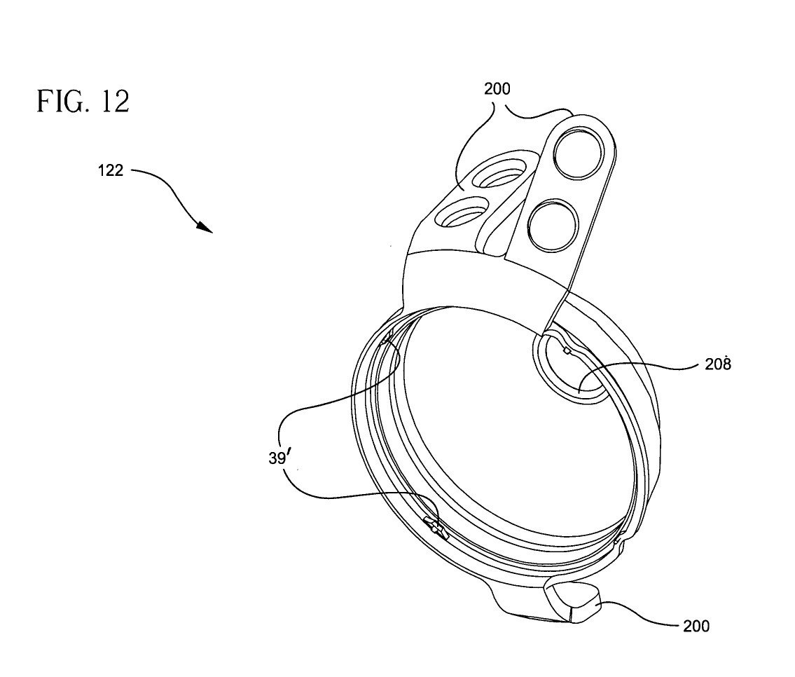

In the preferred embodiment, one or more flanges 200 extend laterally or radially outwardly from the lower end 202 of the adaptor 100. In the preferred embodiment, adaptor 100 includes two flanges 200 which can be shaped in any convenient or desirable manner. Flanges 200 can include any number of holes 204 and can be integrally formed with the remainder of adaptor 200. Alternately, as shown in FIGS. 11 and 12 alternate inserts 120 and 122 have alternate flanges 200. Flanges 200 can be modular and be screwed or clipped onto adaptor 100. Flanges 200 can be provided with grooves 206 which will allow the surgeon to bend the flange further at the time of implantation so as to better conform to the bone remaining in the pelvic area. In addition, adaptor 100 includes protrusions 39′ which can engage the recesses 59 of insert 24 to rotationally lock the bearing 24 within adaptor 100. Likewise, adaptor 100 has indications 120 which engage with the protrusions 39 of shell 22 to prevent rotation therebetween.

In the preferred embodiment, in addition to the taper locking of adaptor 100 and shell 22, a threaded coupling can be utilized to lock the adaptor 100 in position. If such a locking system is used, a polar hole 208 in adaptor 100 and a threaded polar hole 29 in shell 22 engage a threaded retaining member 47. Retaining member 47 has external threads matching the internal threads of screw hole 29. Retaining member 47 includes a flange 49 which is larger than the diameter of polar opening 208 of adaptor 100 so that upon tightening of threaded fastener 47 within the threaded bore 29, adaptor 100 is locked within shell 22 via the tapered interconnection and the threaded connection.

In the preferred embodiment, a plurality of adaptors 100 with flanges located in difference circumferential orientations and extending in different radial lengths may be provided. In addition, flanges or straps 200 can be at various angles to the plane of opening 101 of adaptor 100.

It will be seen that acetabular cup assemblies 20 and adaptors 100 provide a surgeon with a wide range of choices for a pre-operative or an interoperative selection of characteristics of the bearing member of the acetabular cup assembly, with simplicity and lowered cost. Such characteristics include material, size, positioning and orientation. As such, the present invention attains the several objects and advantages summarized above, namely: Accommodates a wide choice of bearing materials in the bearing member of an acetabular cup assembly, while utilizing a common acetabular shell; enables a wide choice of size, position and orientation of the bearing surface of a bearing member selected for assembly with a particular acetabular shell; increases the range of bearing materials, as well as bearing size, positioning and orientation, and renders the choices available in a practical manner for either pre-operative or interoperative selection; allows a surgeon greater latitude in accommodating the needs of different patients while meeting the requirements imposed by various conditions encountered at a particular implant site, and enables appropriate choices to be made interoperatively, as well as pre-operatively; promotes greater accuracy in the replacement of a natural hip joint, with increased economy; provides a surgeon with the ability to make both pre-operative choices and interoperative choices from a wider range of options; enables the securement of a bearing member of selected material within a common acetabular shell, with increased ease and economy, and without complex, specialized instruments; provides an acetabular cup assembly having a bearing member of appropriate bearing material and accurate sizing, positioning and orientation, with economy of manufacture and use, and long-term reliability.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An acetabular cup assembly for a prosthetic hip joint comprising:
an outer shell for attachment to the acetabulum said shell having an inner surface;
a bearing insert portion;
an adaptor having an outer surface for engaging the inner surface of the outer shell and an inner surface for receiving the bearing insert portion, said adaptor having at least one radially extending strap.

2. The acetabular cup assembly as set forth in claim 1 wherein said strap is deformable at least in a direction perpendicular to said radial direction.

3. The acetabular cup assembly as set forth in claim 2 wherein said at least one strap includes a reduced cross-section to facilitate said deformation.

4. The acetabular cup assembly as set forth in claim 3 wherein said reduced cross-section is formed by a groove.

5. The acetabular cup assembly as set forth in claim 1 wherein said inner surface of said shell and said outer surface of said adaptor having complimentary tapered locking surfaces.

6. The acetabular cup assembly as set forth in claim 5 wherein said adaptor and said shell included a threaded connection for lockingly engaging said complimentary tapered surfaces.

7. The acetabular cup as set forth in claim 5 wherein said inner surface of said outer shell has a coupling structure for engaging a complementary locking structure on an outer surface of said bearing element.

8. The acetabular cup assembly as set forth in claim 7 wherein said inner surface and said outer shell has a coupling structure for engaging a complementary locking structure on an outer surface of said bearing component.

9. The acetabular cup assembly as set forth in claim 1 wherein said straps are integral with said adaptor.

10. A kit for resurfacing an acetabulum comprising:
at least one shell having an outer surface for contacting the acetabulum and an inner surface with an open end;
a plurality of flanged inserts having an outer surface for engaging the inner surface of said shell and a rim area having at least one radially extending flange, said flanged insert having an inner surface with an engagement feature;
at least one bearing insert for insertion into said flanged insert and having a locking element for engaging the engagement feature of said flanged insert.

11. The kit as set forth in claim 10 wherein said inner surface of said shell and said outer surface of said adaptor having complimentary tapered locking surfaces.

12. The kit as set forth in claim 10 wherein said adaptor and said shell included a threaded connection for lockingly engaging said complimentary tapered surfaces.

13. The kit as set forth in claim 10 wherein said shell and said flanged inserts each include an internal surface for coupling to said at least one bearing member.

14. A kit of component parts for assembling an acetabular cup assembly having an internal bearing member for selective securement within a shell member interoperatively, the kit comprising:
a plurality of shell members each having an inner cavity;
a plurality of bearing elements insertable within the cavity of the shell member, the shell member having an internal coupling structure compatible with the coupling structure of at least one of the plurality of internal bearing members, at least one adaptor member for insertion into said shell and having a radially extending strap associated therewith for engaging the pelvis.

15. The kit as set forth in claim 14 wherein the adaptor member includes an internal coupling structures identical to the internal coupling structure of said shell member.

16. The kit as set forth in claim 15 wherein the bearing member includes a rib projecting from the bearing member and the internal coupling elements and said shell have a recess for receiving the rib of the bearing member.

17. The kit as set forth in claim 16 wherein the adaptor member includes a tapered external securing surface, and the shell includes a tapered internal securing surface, the external securing surface and the internal securing surface being complementary tapered configurations for interlocking in response to seating engagement of the complementary tapered configurations.

18. The kit as set forth in claim 17 wherein the internal coupling structure on said shell is intermediate ends of said tapered internal surface on said shell.

19. The kit as set forth in claim 13 wherein said straps are selectably attachable to a distal end of said securing element.
